(19) United States
(12) Patent Application Publication
(10) Pub. No.: US 2003/0195476 A1
(43) Pub. Date: Oct. 16, 2003

(54) Umbilical cord care apparatus and method of use

(76) Inventors: Lauree Martin, Iola, TX (US); Anne Christian, Garland, TX (US)

Correspondence Address: HOWREY SIMON ARNOLD & WHITE LLP, 750 BERING DRIVE, HOUSTON, TX, 77057, US

(21) Appl. No.: 10/446,976

(22) Filed: May 28, 2003

Related U.S. Application Data
(63) Continuation of application No. 10/255,294, filed on Sep. 26, 2002.
Application No. 10/255,294 is a continuation of application No. 09/619,840, filed on Jul. 20, 2000, now abandoned.

(51) Int. Cl. A61M 5/315

(52) U.S. Cl. 604/218

(57) ABSTRACT

An apparatus useful for umbilical cord care and method of its use are disclosed. The apparatus provides an injector that is detachably connected to a disposable applicator. The umbilical cord care apparatus may be provided as a kit, particularly suited for use at home.

### FIELD OF THE INVENTION

The present invention relates to an apparatus and method for providing medical care to an infant. More specifically, an apparatus useful for umbilical cord care and method of its use are disclosed. The apparatus may preferably be provided as a kit for use at home.

### BACKGROUND OF THE INVENTION

The umbilical cord is the baby's lifeline from the mother during pregnancy. The cord, formed during the fifth week of gestation, connects the fetus' circulation with the mother's placenta. Through this vascular structure, the fetus receives nutrients such as oxygen, glucose, and protein. By the time the baby is born, the cord is about 2 feet long and ½ inch thick.

Shortly after birth, the umbilical cord is clamped and cut. The remaining structure, known as the umbilical stump, typically protrudes about an inch or less. Normally, the stump turns black within the first few days and falls off after about 2-3 weeks. However, during the period that the umbilical stump is still attached to a new born baby, the stump must be properly cared for to ensure proper healing.

Proper care for the umbilical stump requires that the area be kept clean and dry. Such care typically involves rubbing the area with a cotton swab soaked in rubbing alcohol. In particular, the area at the base of the stump, often tucked under folds of skin, must be thoroughly cleaned. This is usually performed at each diaper change. To keep the stump dry, diapers are typically folded below the stump, exposing it to the air. This routine of umbilical cord care must be maintained until the stump falls off.

If proper care is not performed, the stump or area around the stump may become infected. Symptoms of umbilical stump infections include redness and swelling in the region, oozing or weeping of fluid from the region, and delay of stump disengagement from the baby. Infections may pose serious health concerns for the baby, and often require additional medical treatments.

In spite of its importance, traditional umbilical stump care presents at least two disadvantages. First, the cotton swab may not sufficiently clean the area around the base of the stump. Folds and recesses of skin often limit the access of such objects to the base of the stump. Second, care givers must handle an open bottle of rubbing alcohol around s the baby to soak the cotton swab. This creates a hazardous condition. The alcohol may be inadvertently spilled onto the baby as it is applied to a cotton swab. In addition, the baby, older children, the care giver, or a pet may knock over the bottle of alcohol, splashing it onto the baby.

Thus, there exists a need for a safe and convenient apparatus and method to care for the umbilical stump of a new born baby.

### SUMMARY OF THE INVENTION

This invention relates to an umbilical cord care apparatus and methods of its use. The umbilical cord care apparatus preferably comprises an injector, detachably connected to a disposable applicator.

The injector preferably comprises an open upper end portion, a restricted lower end portion, a plunger, and an interior chamber. The plunger preferably passes through the open upper end portion of the injector and is fluid-tightly disposed within the interior chamber of the injector. The plunger preferably fluid-tightly contacts the walls of the interior chamber and may slide within the interior chamber while maintaining fluid tightness. The restricted lower end portion is preferably designed to draw fluid into the interior chamber of the injector when the restricted lower portion is immersed in a fluid and the plunger is simultaneously pulled away from the restricted lower end portion. The restricted lower end portion is also preferably designed to detachably connect to a disposable applicator. As the plunger moves towards the restricted lower end portion, any fluid contained in the interior chamber will pass through the restricted lower end portion and into the disposable applicator.

The disposable applicator preferably has one or more fluid dispensing openings and is wrapped by a wettable material. This design allows fluid entering the applicator to pass through the applicator and saturate the wettable material. The saturated wettable material may then be applied to an area on and around the umbilical cord stump of an infant.

The umbilical cord care apparatus may also be packaged in suitable container means as a kit. Such a kit may include a plurality of injectors that are empty or pre-filled with a suitable cleansing solvent; a plurality of disposable applicators; and instructions for assembling and using the umbilical cord care apparatus.

The method according to the invention comprises providing an injector of the inventive umbilical cord care apparatus, filling the injector with a suitable cleansing solvent (if the apparatus is not already pre-filled), attaching a disposable applicator to the injector, selecting an infant having an umbilical cord stump, operating the umbilical cord care apparatus to apply the cleansing solvent contained in the injector to the wettable material wrapping the applicator; and using the wettable material to wipe the region on and around the infant's umbilical cord stump.

### DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIG. 1: Refers to a preferred embodiment of a disposable applicator having a conical shape.

FIG. 2: Refers to a preferred embodiment of an injector and applicator.

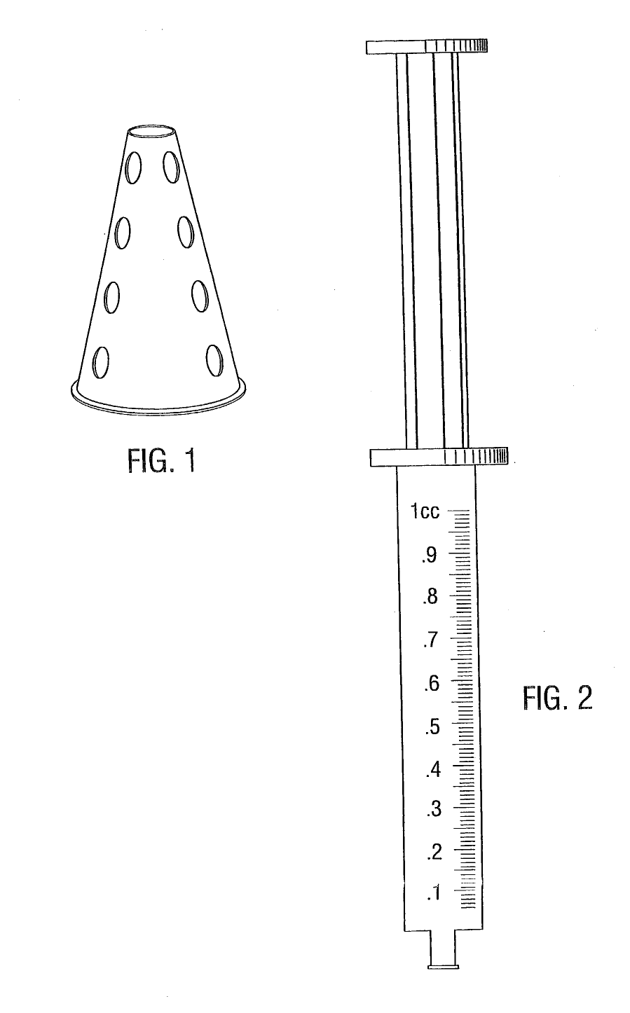

### DETAILED DESCRIPTION OF THE INVENTION

The following description demonstrates preferred embodiments of the invention. It should be appreciated by those of skill in the art that the apparatus and methods of its use have been discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The umbilical cord care apparatus preferably comprises an injector having an open upper end portion, a restricted lower end portion, a plunger, and an interior chamber. The plunger preferably passes through the open upper end portion of the injector and is fluid-tightly disposed within the interior chamber of the injector. The plunger is preferably capable of moving within the interior chamber while maintaining fluid tightness between the plunger and the interior walls of the chamber.

A wide variety of devices may function as an injector in the present invention. The injector is preferably a syringe or syringe-like structure, more preferably a 1 cc, 3 cc, 5 cc, or 10 cc syringe, and most preferably a 3 cc syringe.

The applicator releasably couples to the restricted lower end portion of the injector. At least a partial bore in the applicator is in fluid communication with the interior chamber of the injector when coupled. The applicator typically contains one or more openings. Thus, fluid from the injector may enter the applicator and pass into the wettable material wrapping the applicator. This allows the wettable material to become saturated with the fluid. These openings may be of any size and shape compatible with the present invention. In one embodiment, the applicator contains a longitudinal throughbore. In a preferred embodiment, the applicator contains a longitudinal throughbore and one or more lateral bores or openings. The lateral openings may be fluted.

In an alternative embodiment, the umbilical cord care apparatus may comprise an injector having a de-formable closed upper end portion having an interior chamber, and a restricted lower end portion having an interior chamber. The closed upper end portion is preferably a de-formable bulb. The interior chamber of the upper end portion is preferably enlarged to serve as a fluid reservoir. The interior chamber of the upper end portion is preferably fluidtightly integrated with the interior chamber of the restricted lower end portion. The upper end portion may form a continuous piece with the restricted lower end portion. Alternatively, the upper end portion may be fluid tightly integrated or detachably connected with the lower restricted end portion. The restricted lower end portion is also preferably designed to detachably connect to a disposable applicator (suitable applicators are described above).

The upper end portion is preferably constructed of a de-formable plastic. Such material preferably allows a user to squeeze the upper end portion or bulb with sufficient force to expel the fluid held within the interior chambers of the upper and lower end portions. As force is applied to the upper end portion of the injector, the fluid within the interior chambers moves out of the interior chambers, through the restricted end portion of the injector, and into the applicator. In this manner, fluid may be applied to the umbilical area of an infant.

A key aspect of the present invention is the disposable nature of the inventive apparatus. The injector and the applicator are preferably made of a disposable material, such as a light weight and inexpensive plastic. Alternatively, the applicator itself may be made from a wettable material capable of holding its shape without an artificial support structure, such as high density cotton. In addition, the applicator may be rigid or flexible.

The disposable applicator is typically wrapped in a wettable material. The wettable material is preferably suitable for contact with a baby's sensitive skin. The material should also be useful in cleaning and wiping the area around the baby's umbilical cord stump. The wettable material preferably is cotton or guaze, and most preferably is cotton.

The applicator may be a variety of different sizes. As mentioned above, the applicator should be capable of being detachably connected to the injector. The applicator is preferably threadably connected to the injector, or connected by a snap and groove fitting, a ridge and groove fitting, or a key and a keyway, but may also be connected by any other suitable means.

The shape of the applicator may also vary. However, because the base of the umbilical stump is covered with folds of skin, certain shapes will provide a distinct advantage in accessing these areas. Thus, conical and elongated tips may be particularly advantageous. The applicator is preferably a conical, rectangular, ovoid, or trapezoidal shape, more preferably a conical shape, and most preferably an elongated conical shape. In addition, the tip may comprise a hybrid of shapes, such as a conical shape tapering to a rectangular tip. In using conical shaped applicators, the base of the cone attaches to the restricted lower end portion of the injector while the tapered apex portion of the cone provides improved access to the base of the umbilical stump region.

In a preferred embodiment, the umbilical care apparatus may further comprise features that permit simple assembly and disassembly of the apparatus, easy and safe addition of fluid to the apparatus, easy removal of the disposable applicator, and facilitate the general operation and handling of the apparatus.

The present invention also relates to a method for using an umbilical cord care apparatus. The method comprises providing an injector and applicator of the umbilical cord care apparatus as described above, filing the injector with a suitable cleansing solvent (if the injector is not already pre-filled), attaching the applicator to the filled injector, selecting an infant having an umbilical cord stump, and operating the umbilical cord care apparatus to apply the cleansing solvent contained in the injector to the wettable material on the applicator while using the wettable material on the applicator of the apparatus to wipe the region on and around the umbilical cord stump.

The cleansing solvent may be any soap or disinfectant suitable for contact with a baby's skin. The cleansing solvent is preferably standard rubbing alcohol (˜70% (v/v) isopropyl alcohol).

The present invention further relates to an umbilical cord care kit, comprising in suitable container means the disassembled components necessary to form an umbilical cord care apparatus. Specifically, the kit may include one or more injectors each having an open upper end portion, a restricted lower end portion, a plunger, and an interior chamber; wherein the plunger preferably passes through the open upper end portion of the injector and is fluid-tightly disposed within the interior chamber of the injector; and one or more disposable applicators each capable of being detachably connected to one of the injectors, wherein each applicator has one or more fluid dispensing openings; and wherein each applicator is wrapped with a wettable material.

The injectors provided in such a kit may be empty or pre-filled with a cleansing solvent. The cleansing solvent may be any soap or disinfectant suitable for contact with a baby's skin. The cleansing solvent is preferably standard rubbing alcohol (˜70% (v/v) isopropyl alcohol).

The kit may further comprise additional packaging and instructions for assembling, using, and disassembling the umbilical cord care apparatus.

All of the methods and apparatus disclosed and claimed herein can be made and executed without undue experimentation in light of the present disclosure. While the apparatus and method of this invention have been described in terms of preferred embodiments, it will be apparent to those of skill in the art that variations may be applied without departing from the concept, spirit and scope of the invention. All such similar substitutes and modifications apparent to those skilled in the art are deemed to be within the spirit, scope and concept of the invention.

What is claimed is:

1. An umbilical cord care apparatus comprising:
an injector having an open upper end portion, a restricted lower end portion, a plunger, and an interior chamber; wherein the plunger passes through the open upper end portion of the injector and is fluid-tightly disposed within the interior chamber of the injector; and
a disposable applicator detachably connected to the restricted lower end portion of the injector; wherein the applicator:
has a throughbore; one or more lateral fluid dispensing openings; and is wrapped with a wettable material.

2. The apparatus of claim 1, wherein the injector is a syringe.

3. The apparatus of claim 1, wherein the applicator is conical, rectangular, or trapezoidal in shape.

4. The apparatus of claim 1, wherein the applicator is conical in shape.

5. The apparatus of claim 4, wherein the conical shape tapers to a rectangular end.

6. The apparatus of claim 1, wherein the applicator is rigid.

7. The apparatus of claim 1, wherein the applicator is flexible.

8. The apparatus of claim 1, wherein the applicator is made of a wettable material.

9. The apparatus of claim 1, wherein the one or more openings in the applicator are fluted.

10. The apparatus of claim 1, wherein the wettable material is cotton.

11. The apparatus of claim 2, wherein the disposable applicator is threadably connected to the restricted lower end portion of the injector.

12. A method for using an umbilical cord care apparatus comprising:
a) providing an injector and an applicator according to claim 1;
b) filing the injector with a cleansing solvent, if it is not pre-filled;
c) connecting the applicator to the restricted lower end portion of the injector;
d) selecting an infant having an umbilical cord stump;
e) operating the umbilical cord care apparatus to apply the cleansing solvent contained in the injector to the wettable material wrapping the applicator; and
f) using the wettable material on the applicator of the apparatus to wipe the region on and around the infant's umbilical cord stump.

13. The method of claim 12, wherein the injector is a syringe.

14. The method of claim 12, wherein the applicator is conical, rectangular, or trapezoidal in shape.

15. The method of claim 12, wherein the applicator is conical in shape.

16. The method of claim 15, wherein the conical shape tapers to a rectangular end.

17. The method of claim 12, wherein the applicator is rigid.

18. The method of claim 12, wherein the applicator is flexible.

19. The method of claim 12, wherein the applicator is made of a wettable material.

20. The method of claim 12, wherein the one or more openings in the applicator are fluted.

21. The method of claim 12, wherein the wettable material is cotton.

22. The method of claim 13, wherein the applicator is threadably connected to the restricted lower end portion of the injector.

23. The method of claim 12, wherein the cleansing solvent is rubbing alcohol.

24. An umbilical cord care kit, comprising in suitable container means:
one or more injectors, each having an open upper end portion, a restricted lower end portion, a plunger, and an interior chamber; wherein the plunger passes through the open upper end portion of the injector and is fluidtightly disposed within the interior chamber of the injector;
one or more disposable applicators, each capable of being detachably connected to one of the injectors, wherein each disposable applicator is wrapped with a wettable material and has a plurality of fluid dispensing openings; and
wherein an umbilical cord care apparatus is formed by connecting one of the disposable applicators to the restricted lower end portion of one of the injectors.

25. The kit of claim 24, wherein the injector is a syringe.

26. The kit of claim 24, wherein the applicator is conical, rectangular, or trapezoidal in shape.

27. The kit of claim 24, wherein the applicator is conical in shape.

28. The kit of claim 27, wherein the conical shape tapers to a rectangular end.

29. The kit of claim 24, wherein the applicator is rigid.

30. The kit of claim 24, wherein the applicator is flexible.

31. The kit of claim 24, wherein the applicator is made of a wettable material.

32. The kit of claim 24, wherein the one or more openings in the applicator are fluted.

33. The kit of claim 24, wherein the wettable material is cotton or gauze.

34. The kit of claim 25, wherein the applicator is threadably connected to the restricted lower portion of the injector.

35. The kit of claim 24, wherein one or more injectors are pre-filled with a cleansing solvent.

36. The kit of claim 35, wherein the cleansing solvent is rubbing alcohol.

37. The kit of claim 24, further comprising instructions for assembling, using, and disassembling the umbilical cord care apparatus.

38. An umbilical cord care apparatus comprising:
an injector having a closed upper end portion and an interior chamber, and a restricted lower end portion having an interior chamber, wherein:
the closed upper end portion comprises a de-formable bulb;
the interior chamber within the upper end portion forms a fluid reservoir;
and the interior chamber within the upper end portion is fluid tightly integrated with the interior chamber of the restricted lower end portion;
a disposable applicator detachably connected to the restricted lower end portion of the injector; wherein the applicator:
has a throughbore; one or more lateral fluid dispensing openings; and is wrapped with a wettable material.
